(19) United States
(12) Patent Application Publication
(10) Pub. No.: US 2019/0216366 A1
(43) Pub. Date: Jul. 18, 2019

(54) Health Monitoring System Including Camera for Measuring Body Proportions

(71) Applicants: David R. Hall, Provo, UT (US); Joshua Larsen, Spanish Fork, UT (US); K. Jeffrey Campbell, Spanish Fork, UT (US); Conrad Rosenbrock, Provo, UT (US); Ben Swenson, Lehi, UT (US); Jared Eggett, Lehi, UT (US)

(72) Inventors: David R. Hall, Provo, UT (US); Joshua Larsen, Spanish Fork, UT (US); K. Jeffrey Campbell, Spanish Fork, UT (US); Conrad Rosenbrock, Provo, UT (US); Ben Swenson, Lehi, UT (US); Jared Eggett, Lehi, UT (US)

(21) Appl. No.: 16/012,758

(22) Filed: Jun. 19, 2018

Related U.S. Application Data
(63) Continuation-in-part of application No. PCT/US18/14113, filed on Jan. 17, 2018.

(51) Int. Cl. A61B 5/107; G06T 7/73; G06T 7/60; G06K 9/00; G06T 7/00; H04N 5/33; A61B 5/103; H04N 13/239; H04N 5/232; A61B 5/00; A61B 5/01; A61B 8/08

(52) U.S. Cl. CPC A61B 5/1079; G06T 7/50; G06T 7/60; G06K 9/00369; G06T 7/0012; H04N 5/33; A61B 5/1072; A61B 5/1036; H04N 13/239; H04N 5/23238; A61B 5/0077; A61B 5/015; A61B 5/4362; A61B 8/0866; A61B 5/0035; G06T 2207/30196; G06T 2207/30204; G06T 2207/10048; G06T 2207/10028; G06K 2209/40; G06T 7/73

(57) ABSTRACT

The health-monitoring system includes one or more cameras which collect graphic data and a controller which calculates body proportion measurements based on the graphic data. Specifically, the health-monitoring system measures a user's body parts and their relative positions. These measurements may be combined with those collected by other devices and sensors within the system which conduct other health-related measurements. The other devices and sensors may be housed within a container and a least one of the cameras may also be housed within the container. In some embodiments, the container is a fixture, such as a bathroom medicine cabinet . The camera may be invisible to a user as positioned within the fixture. Consequently, the user may move and behave normally without changes which may occur due to being self-conscious of the camera. Calculations may occur within the controller or on a remote server to which data is transmitted.

### CROSS REFERENCE TO RELATED APPLICATION

This application is a continuation-in-part of copending International Patent Application No. PCT/US18/14113 filed on Jan. 17, 2018 which is hereby incorporated by reference in its entirety.

### BACKGROUND

### Field of the Invention

This disclosure relates to diagnostic devices and telemedicine services and uses thereof.

### Background of the Invention

Many medical devices and diagnostic assays typically used in a clinical setting by trained healthcare professionals are simple enough that a patient could perform them at home with a small amount of training or guidance. However, without training or guidance, many users may operate the medical device or conduct the diagnostic assay incorrectly. This error may render the data collected useless or worse, harmful if the error is not detected.

An option is to visit a healthcare facility to have diagnostic services performed by a trained professional. However, some users may find it inconvenient or impossible to make frequent visits to a healthcare facility. Particularly when the diagnostic measurement is relatively simple to collect with some basic instruction, it may seem an inefficient use of time to acquire such healthcare services, even when the results of a late or improper diagnosis are severe. Consequently, these users may go without the full benefit of certain diagnostic tools which could provide a significant and positive impact on their health.

Another problem for those unable to visit a healthcare facility in person is that some symptoms are difficult to track without interaction with the patient. Examples include behavior, mobility patterns, and generally how well the patient appears. In some instances, daily interaction is optimal to properly assess a patient's health status. This is impractical for many people.

While telemedicine technology has provided remote healthcare services for some situations, it has yet to replace direct interaction between a patient and a trained healthcare provider. Advances are needed to provide remote healthcare services which replace direct human contact.

### BRIEF SUMMARY OF THE INVENTION

We disclose a health-monitoring system that includes multiple medical devices and sensors which a user may implement to collect data relevant to the user's health status. In addition, the health-monitoring system includes at least one camera capable of collecting graphic data. The health-monitoring system may include a controller with a memory and a data transmission receiver which is in electronic communication with the at least one camera. The controller may also be in electronic communication with the medical devices and sensors. The sensors may include a pressure sensor to measure a user's body weight or create a foot pressure map, diagnostic imaging devices, and other devices and sensors which may be housed in the container disclosed in International Patent Application No. PCT/US18/14113.

The controller may store non-transitory computer-readable media which stores instructions that direct a controller to apply an algorithm which calculates at least one body proportion of the user based, at least in part, on the graphic data collected by the at least one camera. The algorithm may also compile health data by combining the at least one body proportion measurement with at least one other health measurement collected from the user.

The cameras may be in a fixed location, mobile, or at least one of both. They may collect a 360° graphic image of the user. In some embodiments, the cameras may include an infrared thermal imaging camera which uses thermal imaging to identify the user's body parts and differentiate them from clothing, shoes, or other inorganic material on the user's person.

In some embodiments, the health-monitoring system includes markers which are placed in defined locations relative to the cameras and within the line of sight of the at least one cameras. Non-transitory computer-readable medium may store instructions which may direct the controller to perform algorithms which use the markers to triangulate the location of the user at the time the graphic data was collected. Accordingly, an accurate assessment of the user's body proportions may be calculated. Alternatively, the user may be instructed to stand on a mark, for example, on the floor or on a pressure sensor placed adjacent to the floor. Because the mark is in a known location relative to the cameras, the graphic data may be used to accurately calculate the user's body proportions.

The health-monitoring system may include a container which may resemble a fixture. The container may house the additional medical devices, sensors, and diagnostic assays which the user may implement to collect additional data. This additional data may be combined with the measurements of the user's body proportions to result in a more complete assessment of the user's health status. An embodiment of the container is disclosed in International Patent Application PCT/US18/14113.

The memory within the controller may store collected data. Non-transitory computer-readable medium may store instructions for comparing the stored data with more current data and to identify changes that may impact the user's health. In some embodiments, the non-transitory computer-readable medium is stored on a remote server. The data may be obfuscated within the controller and then transmitted to the remote server for calculation and storage as described in International Patent No. PCT/US18/13836 filed on Jan. 16, 2018, which is hereby incorporated by reference in its entirety.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings.

FIG. 1 illustrates an embodiment of the disclosed health-monitoring system which multiple cameras triangulate a user's height.

FIG. 2 illustrates an embodiment of the disclosed health-monitoring in which one camera collects data used to calculate a user's height using markers of known locations.

FIG. 3 illustrates an embodiment of the health-monitoring in which a camera collects data used to calculate a user's height as the user stands on a mark.

FIG. 4A illustrates an embodiment of the disclosed health-monitoring system in which multiple cameras collect data used to calculate measurements of a pregnant woman's abdomen.

FIG. 4B illustrates the embodiment shown in FIG. 4A later in the user's pregnancy.

FIG. 5 illustrates an embodiment of the disclosed health-monitoring system in which the user's body is distinguished from clothing using an infrared thermal imaging camera.

FIG. 6A illustrates an embodiment of the disclosed health-monitoring system which detects a hunched posture in the user.

FIG. 6B illustrates the embodiment of FIG. 6A in which the disclosed health-monitoring system detects scoliosis in the user.

### DETAILED DESCRIPTION OF THE INVENTION

Definitions:

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “private” means the data is kept exclusively within the health-monitoring system to which the user has subscribed.

As used herein, “user” means the individual from whom the disclosed health-monitoring system is collecting graphic data.

As used herein, “electronic” means either wired or wireless. For example, the phase “in electronic communication” means either a wired communication between two devices or could mean a wireless communication between devices, such as Wi-Fi.

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

We disclose a heath-monitoring system which collects graphic data and conducts measurements relating to a user's body proportions using the graphic data. The measurements may be used to assess a user's health status. In some embodiments, the health-monitoring system may include one or more cameras, each in a fixed location. In other embodiments, the one or more cameras are mobile. For example, a mobile camera may rotate around the user. A three-dimensional (3D) view of the user may be obtained using either multiple fixed cameras or a mobile camera. In some embodiments, the multiple cameras may include a 3D camera.

Some embodiments include a light detection and ranging (LIDAR) unit. The LIDAR unit may include a photodetector and a laser emitter. In some embodiments, the laser emitter produces a laser beam in the near infrared range. In an example, the laser emitter produces a laser beam with a wavelength between approximately 600 nm and approximately 1000 nm. The LIDAR unit may be located near the one or more cameras. The LIDAR unit may emit a laser beam toward the user when the health-monitoring system is in use. The laser beam may reflect off the user and the backscatter pulses may be detected by the photodetector. Algorithms stored on the non-transitory computer-readable medium may calculate the time that has passed between the time the laser beam was emitted and the time the backscattered light was detected. The distance the user is positioned from the LIDAR unit may be calculated by the algorithm. The LIDAR unit may also be used, along with appropriate algorithms stored on the non-transitory computer-readable medium, to generate a 3D image of the user from which measurements of body proportions may be made.

Some embodiments include an infrared thermal imaging camera. A user may wish to collect body proportion measurements using the health-monitoring system while wearing clothing or shoes. The graphic data that the infrared thermal imaging camera collects may identify the user's body within the graphic data as well as where the shoes or clothing begin. For example, a user wearing high-heeled shoes or a hat may wish to collect a height measurement without removing the shoes or hat. Without an infrared thermal imaging camera, the added height caused by the shoes and hat may be included in the height calculation. Similarly, a user may wish to collect body proportion measurements while wearing a heavy coat. Without an infrared thermal imaging camera, the added fabric of the coat may be included in measurements of the width of the user's waste, shoulders, hips, or other body parts. The infrared thermal imaging camera may produce graphic data which indicates the user's body within the shoes, hat, or coat so that these articles of clothing are not included in the measurements. Consequently, a more accurate measurement may be produced.

The health-monitoring system may include a controller which may include a memory and a data transmission receiver. The controller may be in electronic communication with the one or more cameras through the data transmission receiver. In an example, the health-monitoring system includes one, two, three, four, five, six, or more than six cameras. Each camera may collect graphic data and transmit it to the computer through the data transmission receiver. The controller may then store the graphic data in the memory within the controller.

The controller may include non-transitory computer-readable medium which may apply an algorithm which calculates at least one body proportion of a user based on the graphic data the cameras have collected. Examples of body proportions the algorithm may calculate include a user's height, width across the user's shoulders, and width across the user's pelvis. Angles at which the user carries his/her shoulders and hips relative to a defined plane may be calculated. For example, the angles at which the user carries his/her shoulders or hips relative to a horizontal plane may be calculated. In another example, the angle of a user's spine relative to a vertical plane may be calculated. The health-monitoring system may calculate multiple body proportion measurements and calculate their relative ratios to create data that is relevant to the user's health. Calculations of the user's body proportions may be combined with other health measurements the system collects to create health data which may be used to assess a user's health status or monitor the user's health status over time. Other health measurements include those described in International Patent Application No. PCT/US18/14113 and which may be assessed using devices, sensors, and assays housed in the container described therein. In an example, the health-monitoring system may measure the user's body weight.

The controller may include non-transitory computer-readable medium which may store instructions for creating a 360° view of the user's body. The algorithm may use the 360° view to calculate body proportions that include body parts spanning different sides of the user's body. The 360° view may be created using graphic data collected by a single mobile camera which moves around the user or by compiling data multiple in images collected by multiple fixed cameras.

In some embodiments, the one or more cameras may be located within a fixture. This may be advantageous because the one or more cameras may not be visible to the user. This results in an unobtrusive process where the health-monitoring system may collect data while the user proceeds with his/her normal daily routine. This may be especially important for collecting accurate measurements which may be altered if the user is acutely aware of the data collection and becomes self-conscious. For example, a user may consciously or unconsciously alter his/her posture during graphic data collection causing an inaccurate measurement. In an example, the fixture may be a container resembling a bathroom medicine cabinet as described in International Patent No. PCT/US18/14113. This fixture may include a cabinet door with a partially silvered mirror on it. A camera may be positioned behind the partially silvered mirror. Therefore, the camera may collect graphic data through the mirror while the user does not see the camera.

In some embodiments, the health-monitoring system includes at least two markers that are placed at known distances from a camera. For example, the health-monitoring system may include two, three, four, five, six, or more than six markers. A user may stand within the line of sight of the camera. The markers are also within the line of sight of the camera. The algorithms stored on the non-transitory computer-readable medium may use the known positions of the markers and the graphic data the camera collects to triangulate the position of the user. By identifying the position of the user, the algorithm may then calculate the user's body proportions. For example, by determining where the user is standing relative to the markers, a user who is standing further away from the camera will not be perceived as being shorter than when a user stands closer to the camera.

In another embodiment, the health-monitoring system includes a mark within the line of sight of the camera which indicates where a user may stand or sit while collecting measurements. The mark may be in a defined position relative to the cameras. In an example, the mark may be an area on the floor which is colored differently than the remainder of the floor. The location of the mark relative to each camera may be entered into an algorithm stored on the non-transient computer-readable medium. The camera may collect graphic data including images of the user who is positioned in the proper location relative to the mark. Because the user is located in a known position, the algorithm may use the graphic data to calculate the user's body proportions.

The health-monitoring system includes sensors other than the one or more cameras. These other sensors may collect data that may be relevant to the user's health status. The algorithms within the health-monitoring system may combine data collected by the other sensors with the body proportion calculations.

In some embodiments, the other sensors may include devices which conduct diagnostic medical imaging. In an example, the device may include an ultrasound probe. In another example, the health-monitoring system includes a pressure sensor which may measure the user's body weight. The pressure sensor may also determine a pattern user's foot pressure map which may indicate postural defects, foot complications due to diabetes, and other pathologies.

Referring to the embodiment in which a mark is used to instruct the user where to stand or sit during graphic data collection, the mark may be positioned on the pressure sensor. In this embodiment, the user may simply step onto the pressure sensor which also places the user in an optimal position for the cameras to collect graphic data. The pressure sensor, or other health-monitoring sensors may be in electronic communication with the controller.

The pressure sensor, or any other sensor within the health-monitoring system, may collect the data then transmit it to the controller or to a remote server, either of which may receive the data through the data transmission receiver. Furthermore, either the controller or the remote server may house the non-transitory computer-readable medium discussed herein. In some embodiments, the controller may transmit the data to a remote server for storage and/or access by a healthcare professional.

The controller or the remote server may store the data in the memory for later analysis for and comparison to data collected at another time. In an example, a child's growth may be tracked over time. In another example, the effectiveness of a therapy or the progression of a disease process may be tracked. In yet another example, the user may be pregnant and the health-monitoring system may track the growth of her gestating fetus over time. In the latter example, the non-transitory computer-readable media may store instructions for calculating a body weight of the fetus by first calculating the dimensions of the user's abdomen. This calculation may be combined with an ultrasound image which the user may collect using an ultrasound probe housed within the container.

Some health data may be sensitive, and the user may have concerns about keeping it private when using embodiments of the health-monitoring system which store or analyze data remotely. Accordingly, some embodiments may include non-transitory computer-readable medium which stores instructions for applying an algorithm to transform the graphic data into obfuscated graphic data and for creating an analysis of the obfuscated graphic data. The graphic data may be converted to obfuscated graphic data as described in International Patent Application No. PCT/US18/13836 and decrypted for analysis by the remote server.

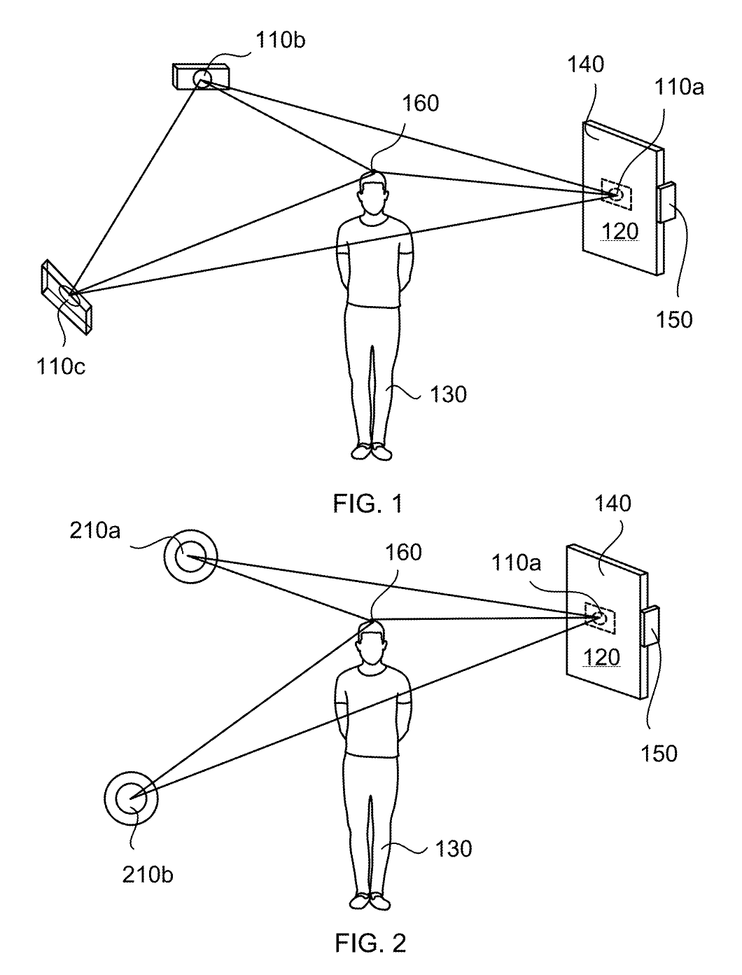

Referring now to the drawings, FIG. 1 illustrates an embodiment of the disclosed healthcare-monitoring system in which cameras 110a, 110b, and 110c are shown collecting graphic data. Camera 110a is shown behind mirror 120, which has a partially silvered cover. Mirror 120 acts as a cover or door on container 140. User 130 stands before mirror 120 while cameras 110a-c collect graphic data including images of the user's body. Because of the partially silvered cover, camera 110a can collect graphic data including images of user 130 without being visible to user 130. While container 140 resembles a traditional medicine cabinet, container 140 is actually the container included in the remote healthcare-monitoring system disclosed in International Patent Application No. PCT/US18/14113. Container 140 houses medical devices, sensors, and diagnostic assays the user may employ to collect health data which may be used to assess the health of user 130 along with the graphic data.

Cameras 110a, 110b, and 110c are positioned in defined locations so that the graphic data they collect may be analyzed and used to measure body proportions using algorithms stored on the non-transitory computer-readable media within controller 150. In this embodiment, the graphic data is being used to triangulate the location of point 160 which represents the height of user 130.

FIG. 2 represents an embodiment of the disclosed healthcare-monitoring system which differs from that of FIG. 1 in that a single camera, camera 110a collects graphic data which include images of markers 210a and 210b. Both of markers 210a and 210b are positioned in defined locations relative to camera 110a so that the relative size of each marker in the graphic image may be used to calculate measurements relating to the body of user 130. Non-transitory computer-readable media on controller 150 may store algorithms which calculate the position of point 160 which is the height of user 130.

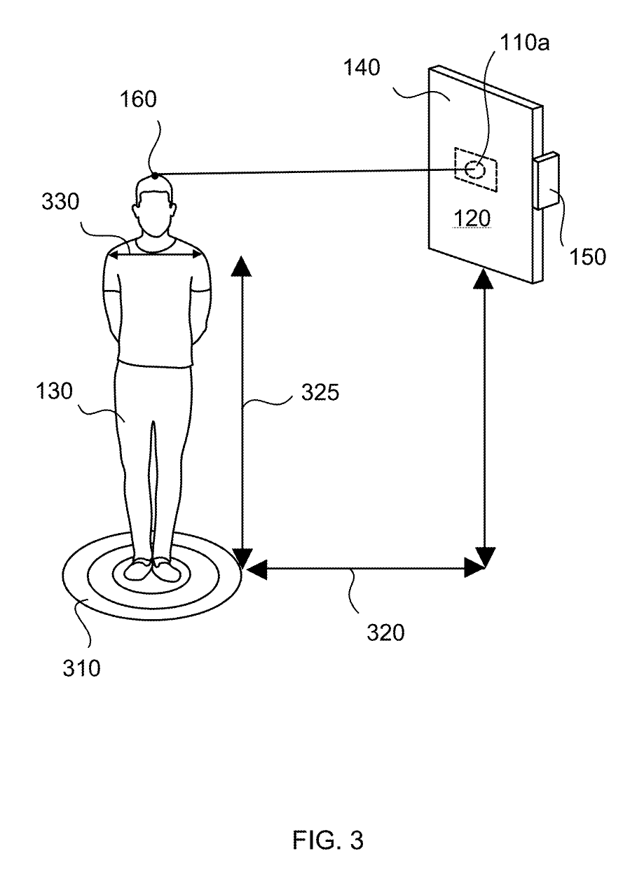

FIG. 3 represents yet another embodiment of the disclosed healthcare-monitoring system which differs from that of FIG. 2 in that user 130 stands on mark 310 which is positioned in a defined location relative to camera 110a. The defined location is distance 320 from container 140. Camera 110a collects graphic data which includes images of user 130 standing on mark 310. Because user 130 is positioned at a known location relative to camera 110a, an algorithm stored on the non-transitory computer-readable media on controller 150 may calculate the position of point 160 which is the height of user 130. Similarly, other bodily measurements may also be calculated. For example, FIG. 3 shows height 325 which is the height of the shoulders of user 130. In another example, this and other embodiments of the health-monitoring system may calculate distance 330 which is the width across the shoulders of user 130 from the left to the right shoulder.

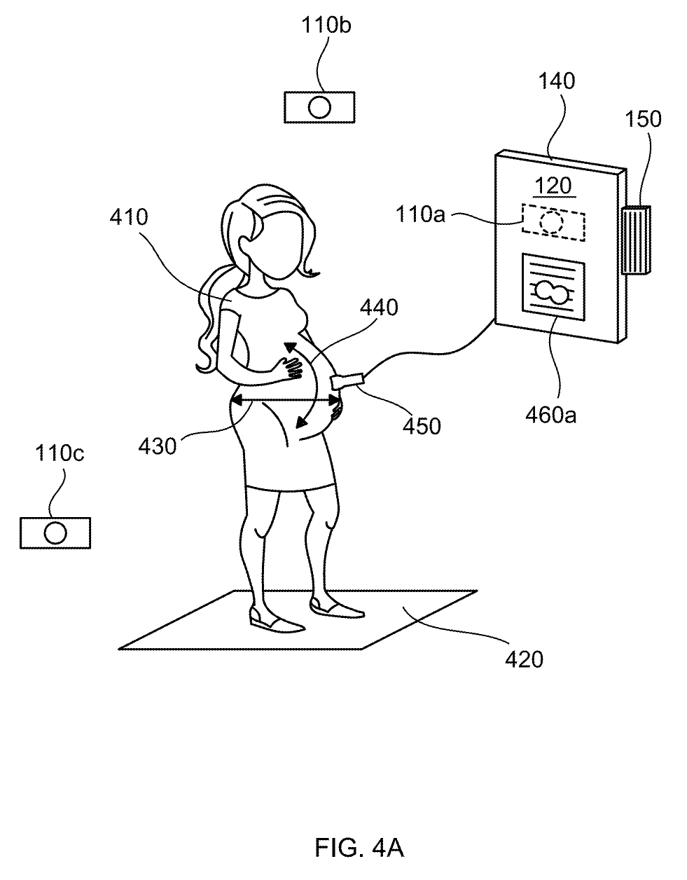

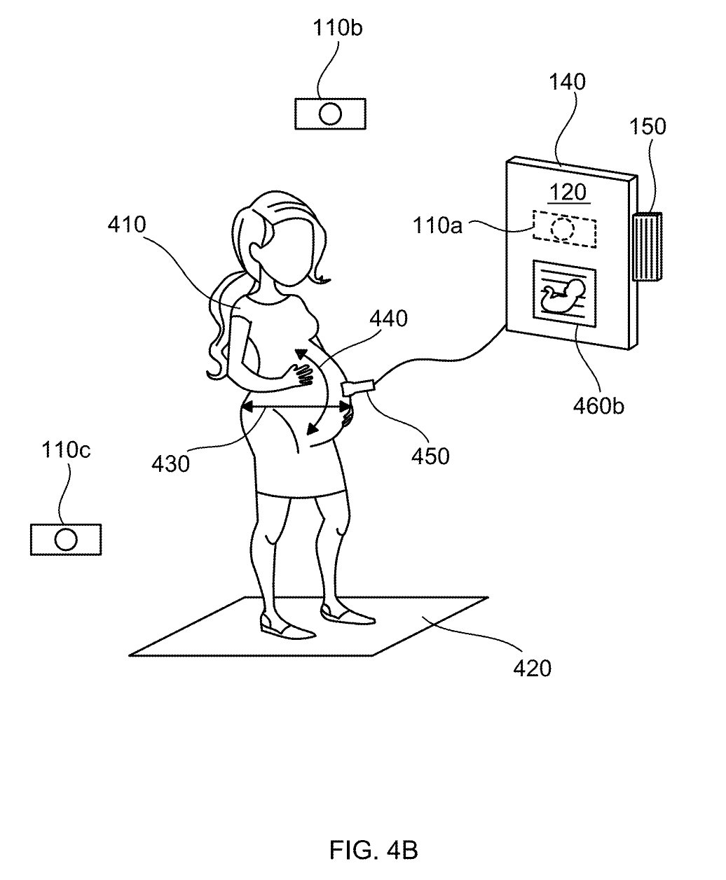

FIG. 4A shows user 410, who is a pregnant woman, collecting measurements using an embodiment of the disclosed healthcare-monitoring system. User 410 has stepped onto pressure sensor 420 which, similar to mark 310 of FIG. 3, is positioned at a defined location relative to cameras 110a-c. Pressure sensor 420 measures the weight of user 410 as she stands on it. Cameras 110a-c collect graphic data from different sides of user 410 and transmit the graphic data to controller 150. In this example, circumference 430, around the waistline of user 410 is measured as well as the distance from the sternum to the pubic bone of user 410. These distances may be used to calculate an estimate of the size of the fetus within user 410.

FIG. 4A also illustrates ultrasound probe 450 which may have been stored in container 140. As user 410 moves ultrasound probe 450 over her abdomen, image 460a is visible on mirror 120. Image 460a shows the fetus within user 410. The image, as well as other measurements, may be stored in a memory within controller 150 for later viewing. Image 460 and the measurements may also be transmitted to a healthcare provider to assess the health of the fetus.

FIG. 4B shows user 410 using the embodiment illustrated in FIG. 4A at a later date in her pregnancy. User 410 repeats the procedure described with regard to FIG. 4A. Image 460b is visible on mirror 120 and may be stored in the memory of controller 150 along with the body weight and other measurements collected during this session. Again, the image and measurements may be transmitted to a healthcare provider who may assess the data and compare them to those collected during the session illustrated in FIG. 4A.

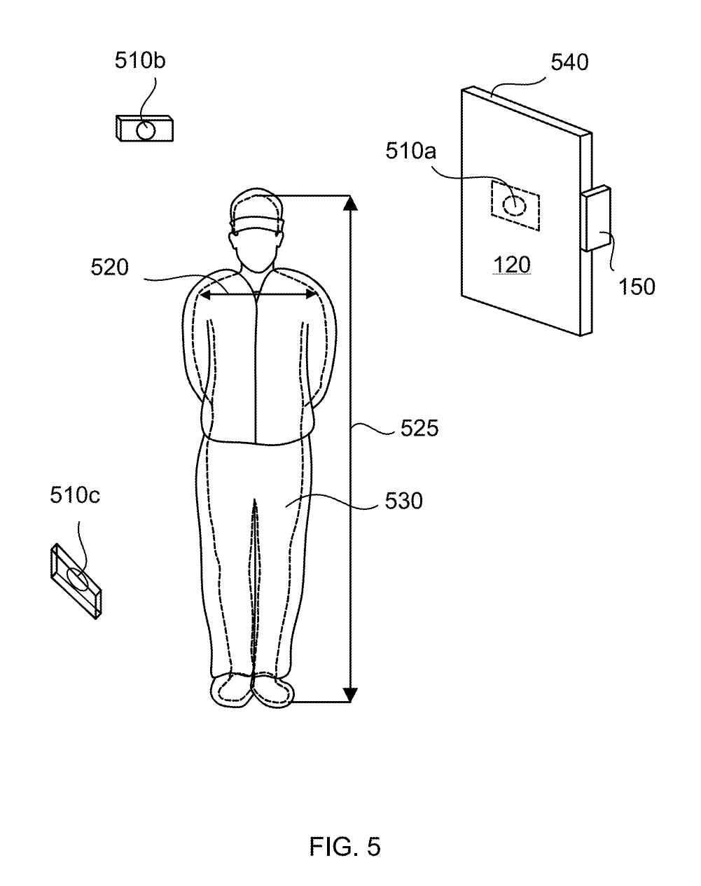

FIG. 5 illustrates an embodiment of the disclosed health monitoring system which includes cameras 510a, 510b, and 510c. These cameras are positioned such that they collect graphic data from different angles. In this embodiment, camera 510a is shown behind the partially silvered mirror in container 540. Cameras 510a-c include infrared thermal imaging technology.

User 530 is wearing clothing which might artificially elevate a measurement across the shoulders due to the bulk of the clothing. In addition, user 530 is wearing a hat which could artificially elevate a measurement of height. Infrared thermal imaging can produce graphic data which shows where the user's body is located within the hat and clothing. The health-monitoring system may use these images to collect measurements including shoulder width 520 as shown by the horizonal arrow and height 525 of user 530 as shown by the vertical arrow. By identifying the body parts of user 530 using infrared thermal imaging, the clothing and hat are not included in the measurements.

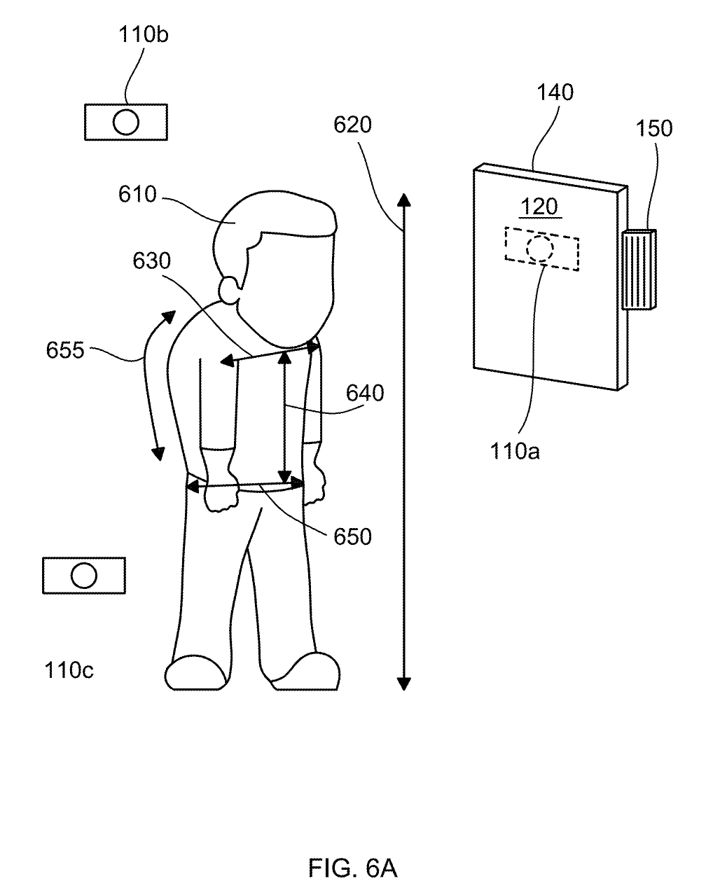

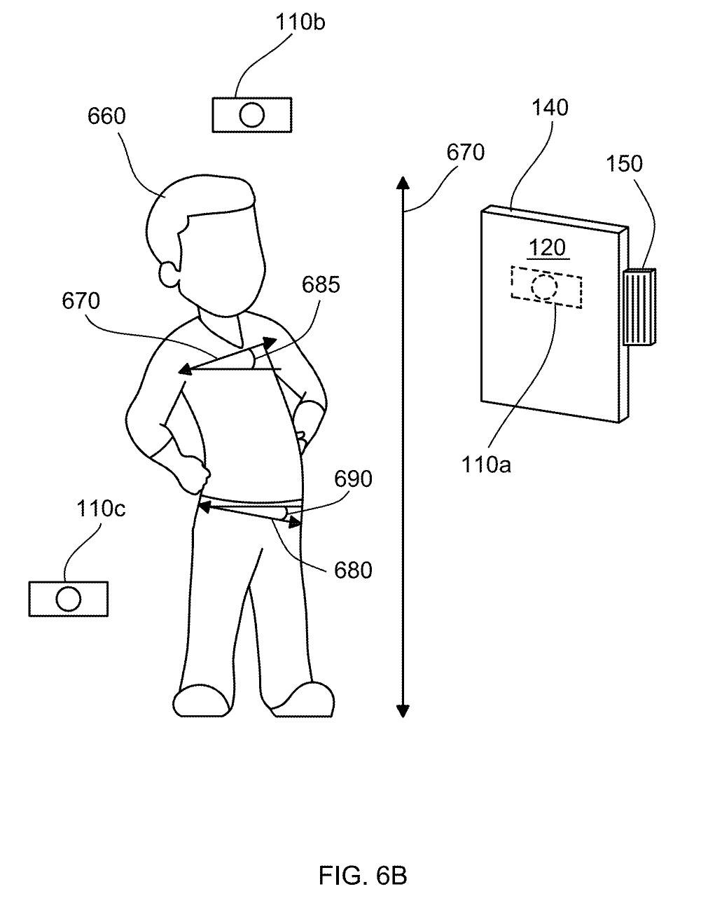

FIG. 6A illustrates user 610 undergoing body proportion measurements using the embodiment of the health monitoring system as first shown in FIG. 1. These measurements include height 620, shoulder width 630, hip width 650, and the distance from the sternum to the hips 640. In addition, the health monitoring system identifies the location of the spine and determines angle 655 of the curvature of the spine relative to vertical. User 610 has osteoporosis which causes the spine to hunch over in a forward bending curvature called kyphosis. Angle 655 can identify a level of kyphosis, similar to determining a Cobb's angle. Computer 150 may record angle 655 and compare it to later measurements. Progression of the degree of curvature may be assessed. In addition, osteoporosis causes reduction in height. By measuring height 620, additional data relating to the progression of osteoporosis may be collected.

FIG. 6B illustrates user 660 undergoing body proportion measurements using the embodiment of the health monitoring system as first shown in FIG. 1. User 660 suffers from scoliosis with the accompanying kyphosis (curvature of the spine). In addition to measuring shoulder width 670 and hip width 680, the angle of these measurements relative to a horizontal plane is being calculated. Angle 685 is the angle of the shoulders relative to a horizontal plane and angle 690 is the angle of the hips relative to a horizontal plane. These measurements may be used to assess the degree of spinal curvature in user 660.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure. Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A health-monitoring system comprising:
at least one camera;
a container, the container comprising a plurality of units, each of the plurality of units housing at least one medical product, diagnostic test, or medical device, wherein each of the at least one medical product, diagnostic test, or medical device is configured to collect a health measurement;
a controller, the controller comprising a memory, and a data transmission receiver, wherein the controller is in electronic communication with the at least one camera, and wherein the controller is configured to receive graphic data from the at least one camera; and
non-transitory computer-readable media comprising instructions stored thereon that, when executed by the controller, cause the controller to apply an algorithm to calculate at least one body proportion of a user based, at least in part, on the graphic data collected by the at least one camera, and for calculating health data by combining the at least one body proportion with the health measurement.

2. The health-monitoring system of claim 1, wherein the at least one camera comprises a 3D camera.

3. The health-monitoring system of claim 1, further comprising a light detection and ranging (LIDAR) unit.

4. The health-monitoring system of claim 1, further comprising at least two markers disposed in defined locations, wherein the non-transitory computer-readable media further comprises instructions for triangulating a position of the user based on the defined positions of the at least two markers and on graphic data comprising images which comprise the at least two markers.

5. The health-monitoring system of claim 1, further comprising a mark on a floor, wherein the mark is a defined distance from the at least one camera.

6. The health-monitoring system of claim 5, further comprising a pressure sensor, wherein the mark is disposed on the pressure sensor.

7. The health-monitoring system of claim 1, wherein the at least one body proportion comprises the user's height.

8. The health-monitoring system of claim 1, wherein the at least one health measurement comprises the user's body weight.

9. The health-monitoring system of claim 8, further comprising a pressure sensor disposed on a floor within a field of view of the at least one camera, wherein the pressure sensor is in electronic communication with the controller, and wherein the pressure sensor is configured to conduct a measurement of the user's body weight and to transmit the measurement to the controller.

10. The health-monitoring system of claim 1, wherein the at least one camera comprises an infrared thermal imaging camera.

11. The health-monitoring system of claim 10, wherein the non-transitory computer-readable media further comprises instructions for identifying the user's body within the graphic data base on an infrared measurement collected by the infrared thermal imaging camera.

12. The health-monitoring system of claim 1, wherein the at least one camera comprises at least two cameras.

13. The health-monitoring system of claim 12, wherein the non-transitory computer-readable media further comprises instructions for compiling a 360° image of the user using the graphic data.

14. The health-monitoring system of claim 1, wherein the at least one body proportion comprises a plurality of body proportions, and wherein the non-transitory computer-readable media further comprises instructions for calculating ratios of at least two of the plurality of body proportions.

15. The health-monitoring system of claim 1, wherein the memory is configured to store the at least one body proportion, and wherein the non-transitory computer-readable media further comprises instructions for calculating a change in the at least one body proportion over time.

16. The health-monitoring system of claim 15, wherein the at least one body proportion comprises an abdominal region of the user, and wherein the non-transitory computer-readable media further comprises instructions for calculating a change in body weight of a fetus within the user.

17. The health-monitoring system of claim 1, wherein the at least one health measurement comprises diagnostic medical imaging.

18. The health-monitoring system of claim 1, wherein the non-transitory computer-readable media further comprises instructions for applying an algorithm to transform the graphic data into obfuscated graphic data and for creating an analysis of the obfuscated graphic data.

19. The health-monitoring system of claim 1, wherein the at least one camera is disposed within a fixture.

20. The health-monitoring system of claim 19, wherein the fixture comprises a partially silvered mirror, and wherein the partially silvered mirror is disposed in front of a lens of the at least one camera.
